(19) United States
(12) Patent Application Publication
(10) Pub. No.: US 2008/0234828 A1
(43) Pub. Date: Sep. 25, 2008

(54) Implant

(75) Inventors: Cecile Wagner, Tuttlingen (DE); Richard Zeller, Tuttlingen (DE); Jens Beger, Tuttlingen (DE)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 12/077,189

(22) Filed: Mar. 17, 2008

(30) Foreign Application Priority Data
Mar. 19, 2007 (DE) 10 2007 014 285.6

(51) Int. Cl. A61F 2/44

(52) U.S. Cl. 623/17.16

(57) ABSTRACT

In order to improve an implant for replacing a vertebral body or for insertion into an intervertebral cavity between adjacent vertebral bodies of a human or animal spinal column, with a first contact face for resting on a joint face of a first vertebral body and with a second contact face for resting on a joint face of a second vertebral body, the implant being at least partially produced from an X-ray transparent material and comprising at least one X-ray marker in such a way that it can be produced more simply, it is proposed that the at least one X-ray marker is configured in the form of an at least partial marker coating, which contains metal as the marker material, of a side face of the implant, which is inclined relative to the first and/or the second contact face by an angle of inclination.

The present disclosure relates to the subject matter disclosed in German application number 10 2007 014 285. 6 of Mar. 19, 2007, which is incorporated herein by reference in its entirety and for all purposes.

### BACKGROUND OF THE INVENTION

The present invention relates to an implant for replacing a vertebral body or for insertion into an intervertebral cavity between adjacent vertebral bodies of a human or animal spinal column, with a first contact face for resting on a joint face of a first vertebral body and with a second contact face for resting on a joint face of a second vertebral body, the implant being at least partially produced from an X-ray transparent material and comprising at least one X-ray marker.

In a conventional treatment of a spinal disc incident, a spinal disc arranged between adjacent vertebral bodies in an intervertebral cavity, also called a spinal disc compartment, is completely or partly removed and the spinal column segment comprising the two adjacent vertebral bodies is fused. For this purpose, a spacer, which is also called a “cage”, is generally inserted into the spinal disc compartment. On the one hand, a height of the segment, in other words a spacing between joint faces of adjacent vertebral bodies can thus be obtained and, on the other hand, the two vertebral bodies can be rigidly connected to one another, so that bone in particular can also grow in the spinal disc compartment.

In recent years, intervertebral implants in the form of spacers made from X-ray permeable materials have also become known. It is thus possible to make a growth of bone through the implant visible by radiographs or CT scans. The disadvantage in X-ray transparent materials of intervertebral implants or else implants for replacing a vertebral body is that to check a positioning of the implants, additional X-ray markers have to be provided on the implant, which allow a position of the implant after an implantation to be checked, for example, on an X-ray image. It is laborious and expensive to configure X-ray markers of this type on the implant.

Therefore, it would be desirable to provide an implant of the type described at the outset which can be produced more simply.

### SUMMARY OF THE INVENTION

It is suggested in an implant of the type mentioned at the outset that the at least one X-ray marker is configured in the form of an at least partial marker coating, which contains metal as the marker material, of a side face of the implant, which is inclined relative to the first and/or second contact face by an angle of inclination.

It is clearly easier and more economical to apply a marker coating than to provide pins or balls on the implant in the known manner. In addition, the marker coating, depending on the selection of a coating material, can also improve the growing on or in of bones onto or into the implant. Furthermore, a position of the marker coating of a side face of the implant can be optimally seen, for example, on a radiograph of a spinal column in a side view. In particular, the marker coating is configured in such a way that it only partially covers one or more side faces of the implant. Obviously, a complete side face may also be covered, so this, as a whole, is used as an X-ray marker.

The implant becomes particularly economical to produce if the X-ray transparent material is a plastics material. Plastics materials also have the advantage that a compressive strength approximating bones can be adjusted, in other words a certain elasticity of the implant can be predetermined in comparison to an implant produced from metal.

The plastics material preferably is, or contains, polyether ether ketone (PEEK). A plastics material of this type is already approved as an implantation material.

Favourably, the angle of inclination has a value in the range of 45° to 135°. Thus, defined structures predetermined in particular by the marker coating can be recognised particularly well in a radiograph of the spinal column from the side.

It is advantageous if the angle of inclination has a value in a range from 70° to 110°. The angle of inclination is preferably 900. This means that one or more side faces may also be configured perpendicularly to the contact faces.

In order, in particular, to be able to clearly indicate an orientation of the implant on a radiograph, it is favourable if the at least one X-ray marker is configured in the form of a regular or irregular pattern. Moreover, the production of the implant is facilitated in this way.

The at least one X-ray marker is preferably configured in the form of at least one letter, number and/or code. Line codes, for example, bar codes would also be conceivable, in particular, with which, on the one hand, a position and/or orientation of the implant in the body can be determined and, on the other hand, even after implantation, information can be read for example at any time about the manner, type, batch number, producer and/or material, from which the implant is produced, or the like. The X-ray marker can thus be simultaneously used as an optical memory element for implant characteristics.

In order to be able to still better and more precisely determine a position of the implant in the body of the patient, a plurality of X-ray markers are advantageously provided.

It is advantageous if the plurality of X-ray markers are provided at least partially on different side faces and/or different sides of the implant. Thus, a position and/or an orientation of the implant in the body of the patient can always be precisely determined regardless or substantially regardless of a recording direction transversely to a longitudinal axis of the body.

It is particularly favourable if the marker material contains titanium. The risk to the patient of a rejection of an implant can thus be minimised.

It is particularly favourable if the marker coating is produced from pure titanium. A rejection reaction can thus be practically completely ruled out.

The coating may be applied particularly easily to an X-ray permeable material, in particular, if it is produced by cold gas spraying. Particularly low process temperatures allow metal coatings to also be applied to plastics materials with a low melt temperature or in a low flow temperature range.

So that an X-ray permeable material, for example a plastics material, is not damaged by the application of the marker coating, it is advantageous if a particle speed of powder particles of the marker material applied by cold gas spraying is in a range of 600 m/s to 1000 m/s. If the powder particles of the marker material are moved at such speeds onto the implant to be coated, an optimal connection of the powder particles with the X-ray transparent material is made possible and a lasting connection of the coating and the implant is ensured.

In order, in particular, to not damage an implant produced from a plastics material during the application of the marker coating, it is favourable, if a jet temperature during the cold gas spraying is in a range from 250° C. to 700° C. The jet temperature is preferably in a range from 250° C. to 500° C.

The marker coating preferably has a thickness in a range from 0.1 mm to 0.3 mm. Thus, an adequate X-ray-impermeability can be produced, so the marker coating can easily be recognised on a radiograph.

In order to prevent the implant moving relative to the vertebral bodies before a final fusion, it is advantageous if the first and/or the second contact face are structured.

It is particularly advantageous if the first and/or the second contact face have projections and/or recesses. These may, in particular, form a structuring and preferably be configured in the form of channels, toothings, raised portions or the like, which can at least partially engage in a surface of a vertebral body to prevent a relative movement before a final fusion of the adjacent vertebral bodies to one another.

Preferably, it may be provided that the implant comprises a first contact element and a second contact element, that the first contact element comprises the first contact face and that the second contact element comprises the second contact face. By means of a suitable selection of the contact elements both with regard to the form thereof and also the material from which they are produced, optimised implants can be configured for any purpose of use, in particular both as an intervertebral implant, in other words in the form of an artificial spinal disc, or as a vertebral body implant for complete or partial replacement of a degenerated and resected vertebral body.

An interlocking of adjacent vertebral bodies can be achieved particularly easily if the first and second contact element are immovably connected to one another.

A stability of the implant can be further increased if it comprises a base body, which comprises the first and the second contact element and is configured in one piece.

The growth of bone into the implant is facilitated and accelerated if the base body is partially hollow and/or has at least one recess.

It is advantageous for the configuration of artificial vertebral discs, in particular, if the first and the second contact element are movably mounted relative to one another.

The first and the second contact element are preferably pivotably and/or displaceably mounted relative to one another. A pivotable mounting may be provided, in particular, about one pivot axis. A pivotable mounting about an articulation centre would also be conceivable, for example, an articulated mounting in the form of a ball and socket joint could be provided, for example.

To improve growth into and onto the implant on a vertebral body, it may be advantageous if the first and/or the second contact face are at least partially provided with an osteointegrative coating. The first and/or the second contact face are preferably completely provided with an osteointegrative coating. This coating may, in particular, be applied simultaneously with the marker coating if these are produced from the same material. The manufacturing, in particular of implants, which have an osteointegrative coating, is simplified thereby.

The growing in of bone and therefore a permanent connection of the implant to the adjacent vertebral body is facilitated if the osteointegrative coating is porous.

To avoid rejection reactions, it is advantageous if the osteointegrative coating is a coating containing titanium.

However, it is particularly advantageous if the osteointegrative coating is a pure titanium coating. A coating of this type has particularly good properties for the growing on and in of bone into the implant.

The osteointegrative coating can be applied particularly easily, in particular on implants produced from plastics materials if it is produced by cold gas spraying.

In addition, it has the same advantages as a marker coating produced by cold gas spraying.

For a stable, lasting coating which facilitates the growing in of bone, it is advantageous if the osteointegrative coating has a thickness in the range from 0.2 mm to 0.5 mm.

It is favourable if the osteointegrative coating is thicker than the marker coating. This configuration in particular allows the implant to be coated completely, for example to be provided with a thick osteointegrative coating on the contact faces, and all the other side faces of the implant to be provided with a thinner marker coating. A bone which is grown into the implant could then nevertheless be seen in a radiograph.

The following description of preferred embodiments of the invention is used, in conjunction with the drawings, for closer explanation.

### BRIEF DESCRIPTION OF THE DRAWINGS

FIG. 1: shows a plan view of an implant inserted in an intervertebral cavity; and

FIG. 2 shows a schematic view of a radiograph with the viewing direction in the direction of the arrows in FIG. 1.

### DETAILED DESCRIPTION OF THE INVENTION

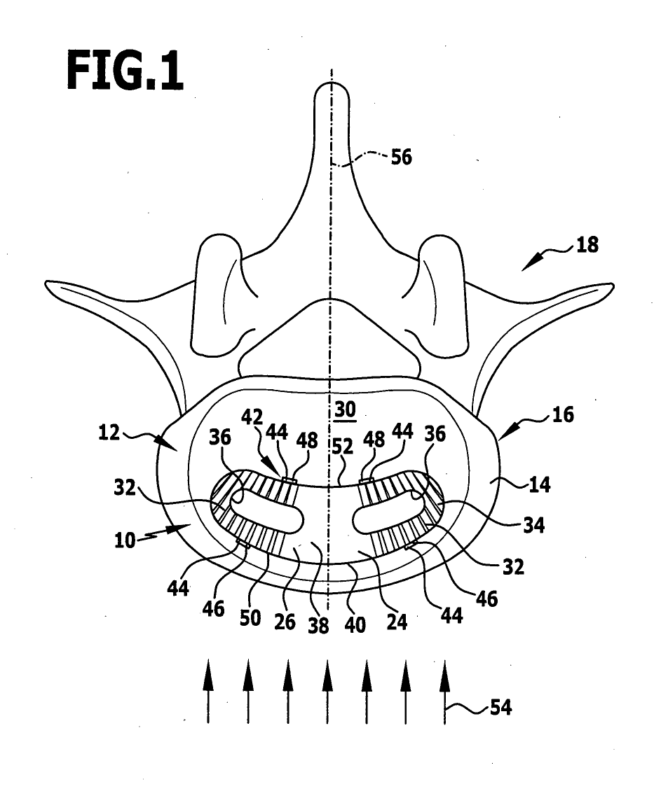

An implant provided as a whole with the reference numeral 10 is shown by way of example in FIG. 1 for insertion into an intervertebral cavity 12 between adjacent vertebral bodies 14 of two vertebrae 16 of a spinal column 18 of a patient.

The implant 10 comprises a first contact element 20 and a second contact element 22, which are immovably connected to one another, specifically by a base body 24, which comprises the first and second contact element 20 and 22 and is produced in one piece from the X-ray transparent material. The X-ray transparent material is a plastics material, preferably polyether ether ketone (PEEK). The first contact element 20 comprises a first contact face 26 for resting on a joint face of a first vertebral body 14. The second contact element 22 comprises a second contact face 28 for resting on a joint face 30 of a second vertebral body 14.

Both the first contact face 26 and the second contact face 28 are structured. In each case, they have two toothing regions 32, with striated teeth 34 directed away from the respective contact face 26 or 28.

Furthermore, the implant 10 which is approximately kidney-shaped in plan view has two slot-like openings 36, which pass through both contact faces 26 and 28 and define a passage direction which is oriented perpendicular to the contact faces 26 and 28, which run parallel to one another. The base body 24 is therefore partially hollow or has at least one recess in the form of an opening 36.

The first contact face 26 and the second contact face 28 are in each case provided with an osteointegrative coating 38. This coating is preferably porous. Furthermore, it may contain titanium or be configured in the form of a pure titanium coating. It has a thickness in a range of 0.2 mm to 0.5 mm, preferably in a range of 0.3 mm to 0.4 mm. The coating 38 is produced by cold gas spraying, a particle speed of the powder particles applied by cold gas spraying being in a range from 600 m/s to 1000 m/s during production. Furthermore, a jet temperature during the cold gas spraying is in a range from 250° C. to 700° C., preferably in a range from 250° C. to 500° C.

The osteointegrative coating facilitates the growing on or in of bone on or in the implant 10.

The implant 10, because of its kidney-shaped form only has a single continuous side face 40, which in the embodiment shown in FIG. 1 is oriented perpendicularly or substantially perpendicularly to the contact faces 26 and 28. In other words, it is inclined relative to the contact faces 26 and 28 by an angle of inclination which has a value of 90°. The side face 40—a plurality of side faces may also be provided—specifically depending on the form of the implant 10, may, however, also be inclined relative to the contact faces 26 and 28 by an angle of inclination, which has a value in a range from 45° to 135°, preferably in a range from 70° to 110°.

The implant 10 also comprises an X-ray marker 42 which is configured in the form of a marker coating 44, which comprises four strips 46 or 48, the two strips 46 being applied running perpendicularly to the contact faces 26 and 28 on a side 50 of the implant 10 facing the front, and the two strips 48 being applied on a side 52 facing the rear. A spacing between the two strips 46 on the side 50 is greater than a spacing between the strips 48 on the side 52. All the strips 46 and 48 extend perpendicularly to the contact faces 26 and 28. The X-ray marker 42 is therefore configured in the form of a regular pattern. In the present case, the X-ray marker 42 is configured in the manner of a code, the strips 46 and 48 forming components of the code. As an alternative to the strips 46 and 48, letters, numbers or any other geometric figures may also be applied directly as a coating. Obviously, a plurality of X-ray markers may be provided, it also being possible for each of the strips 46 and 48 to be considered as individual X-ray markers.

The X-ray marker 42 is preferably formed from a metal, in particular from a metal containing titanium. In the embodiment shown in the Figures, the marker coating is produced from pure titanium. It is furthermore applied by cold gas spraying, a particle speed of the powder particles of the marker material applied by cold gas spraying being in a range from 600 m/s to 1000 m/s. A jet temperature during cold gas spraying of the marker coating 44 is in a range from 250° C. to 700° C., preferably in a range from 250° C. to 500°. A thickness of the marker coating 44 is in a range from 0.1 mm to 0.3 mm. The marker coating 44 is preferably thinner than the osteointegrative coating 38.

The marker coating 44 and the osteointegrative coating 38 may be applied simultaneously in terms of production. Overall this simplifies the production of the implant as X-ray markers in the form of pins or balls do not have to be inserted retrospectively into the base body of the implant as is the case in known implants which have X-ray markers.

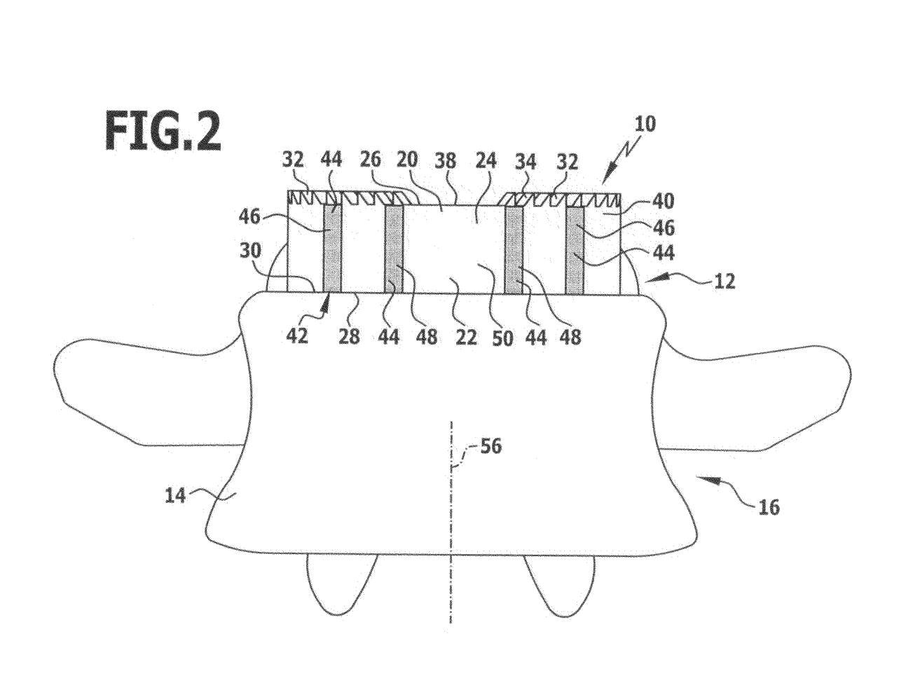

As shown schematically in FIG. 2, the marker coating 44 is visible in the radiograph when acted upon by X-rays 54. As only a small part of the side face 40 is covered with the marker coating 44, a large part of the base body 24 remains uncovered, so the growth of bone into the implant 10 can easily be followed during the further course of healing by taking X-ray images. A position and also an orientation of the implant 10 in the intervertebral cavity 12 can be determined from the position of the strips 46 and 48 in the radiograph. In the frontal image shown in FIG. 2, the strips 48 are located between the strips 46, as the schematically shown radiograph shows a projection of the spinal column in a plane perpendicular to the median plane 56.

1. Implant for replacing a vertebral body or for insertion into an intervertebral cavity between adjacent vertebral bodies of a human or animal spinal column, with a first contact face for resting on a joint face of a first vertebral body and with a second contact face for resting on a joint face of a second vertebral body, the implant being at least partially produced from an X-ray transparent material and comprising at least one X-ray marker, wherein the at least one X-ray marker is configured in the form of an at least partial marker coating, which contains metal as the marker material, of a side face of the implant, which is inclined relative to the first and/or the second contact face by an angle of inclination.

2. Implant according to claim 1, wherein the X-ray transparent material is a plastics material.

3. Implant according to claim 2, wherein the plastics material is or contains polyether ether ketone (PEEK).

4. Implant according to claim 1, wherein the angle of inclination has a value in a range from 45° to 135°.

5. Implant according to claim 4, wherein the angle of inclination has a value in a range from 70° to 110°.

6. Implant according to claim 1, wherein the at least one X-ray marker is configured in the form of a regular or irregular pattern.

7. Implant according to claim 1, wherein the at least one X-ray marker is configured in the form of at least one letter, number and/or code.

8. Implant according claim 1, wherein a plurality of X-ray markers are provided.

9. Implant according to claim 7, wherein the plurality of X-ray markers are provided at least partially on different side faces and/or different sides.

10. Implant according to claim 1, wherein the marker material contains titanium.

11. Implant according to claim 1, wherein the marker coating is produced from pure titanium.

12. Implant according to claim 1, wherein the marker coating is produced by cold gas spraying.

13. Implant according to claim 12, wherein a particle speed of powder particles of the marker material applied by cold gas spraying is in a range from 600 m/s to 1000 m/s.

14. Implant according to claim 13, wherein a jet temperature during cold gas spraying is in a range from 250° C. to 700° C.

15. Implant according to claim 1, wherein the marker coating has a thickness in a range from 0.1 mm to 0.3 mm.

16. Implant according to claim 1, wherein the first and/or the second contact face are structured.

17. Implant according to claim 1, wherein the first and/or the second contact face have projections and/or recesses.

18. Implant according to claim 1, wherein the implant comprises a first contact element and a second contact element, wherein the first contact element comprises the first contact face and wherein the second contact element comprises the second contact face.

19. Implant according to claim 18, wherein the first and the second contact element are immovably connected to one another.

20. Implant according to claim 18, further comprising a base body which comprises the first and the second contact element and is configured in one piece.

21. Implant according to claim 20, wherein the base body is partially hollow and/or has at least one recess.

22. Implant according to claim 18, wherein the first and the second contact element are mounted so as to be movable relative to one another.

23. Implant according to claim 22, wherein the first and the second contact element are mounted so as to be pivotable and/or displaceable relative to one another.

24. Implant according to claim 1, wherein the first and/or the second contact face are at least partially provided with an osteointegrative coating.

25. Implant according to claim 24, wherein the osteointegrative coating is porous.

26. Implant according to claim 24, wherein the osteointegrative coating is a coating containing titanium.

27. Implant according to claim 26, wherein the osteointegrative coating is a pure titanium coating.

28. Implant according to claim 24, wherein the osteointegrative coating is produced by cold gas spraying.

29. Implant according to claim 24, wherein the osteointegrative coating has a thickness in a range from 0.2 mm to 0.5 mm.

30. Implant according claim 24, wherein the osteointegrative coating is thicker than the marker coating.
